(19) United States
(12) Patent Application Publication
(10) Pub. No.: US 2009/0269788 A1
(43) Pub. Date: Oct. 29, 2009

(54) METHOD FOR DETECTING MICROORGANISM

(75) Inventor: Pradip Dahyabhai Patel, Wiltshire (GB)

Correspondence Address: INTELLECTUAL PROPERTY / TECHNOLOGY LAW, PO BOX 14329, RESEARCH TRIANGLE PARK, NC, 27709, US

(73) Assignee: Alaska Food Diagnostics Limited, Wiltshire (GB)

(21) Appl. No.: 12/296,068

(22) PCT Filed: Apr. 5, 2007
(86) PCT No.: PCT/GB2007/001292

§ 371 Date: Apr. 14, 2009

(30) Foreign Application Priority Data
Apr. 5, 2006 (GB) 0606822.5

(51) Int. Cl. G01N 33/543; C12Q 1/04; C12Q 1/48; C12Q 1/37; G01N 33/569

(52) U.S. Cl. 435/7.32; 435/34; 435/15; 435/23; 435/7.35; 435/7.37

(57) ABSTRACT

A method for detecting a microorganism in a sample containing or suspected of containing said microorganism, said method comprising: i) contacting said sample with a binding agent for said microorganism, wherein the binding agent is immobilised on a support, and allowing the binding agent to bind microorganism to form an immobilised complex; ii) separating the sample from the immobilised complex; iii) contacting the support with a liquid medium and a reagent which removes which eliminates, inactivates or inhibits a contaminant that may interfere with a microorganism detection assay; and iv) detecting microorganisms retained on the support using said microorganism detection assay.

The present invention relates to methods for separating microorganisms and in particular bacteria from samples. Such methods are useful as a preliminary step for example in a detection or quantitation step, for example when seeking to identify the presence of bacteria in samples for example of consumer products including food samples or clinical samples. Kits for use in the methods form a further aspect of the invention.

The detection of microorganisms such as bacteria or fungi is important in a wide variety of detection, diagnostic and health fields. For instance, the detection of microorganisms in consumer goods such as food, medicaments or cosmetic preparations is an important procedure to ensure quality control and public safety. Detection of microorganisms in samples such as clinical samples or samples collected for public health purposes may be important for diagnostic or health protection purposes. There is a need to detect even low levels of bacteria in these instances, in particular where the bacteria are pathogenic organisms, such as Salmonella, Listeria and E. coli such as toxigenic E. coli (and in particular the highly pathogenic strain E. coli 0157).

Classical culture techniques in which the presence of microorganisms is investigated by plating out the samples and allowing cultures to grow can take long periods of time. If potential colonies can be identified after a suitable period of time, confirmation of the identity of the colony for example using biochemical identification techniques and ultimately serology, must be carried out.

This process can take anything up to 5 days to complete. Delays of this type are unacceptable in situations where, for example, the substrate comprises a degradable foodstuff which has a limited shelf life.

Alternative commercial techniques (e.g. ELISAs, DNA probes and impedance) can detect the presence of microorganism at levels as low as approximately 105-10′ cfu per ml, which means that they still require at least 24 hours, but more often 48 hours, of cultural enrichment prior to rapid detection of the organism (Patel & Williams, (1994) “Evaluation of commercial kits and instrinnents for the detection of foodborne pathogens and bacterial toxins” in “Rapid Analysis Techniques in Food Microbiology”. Ed. P. D. Patel. Blackie Academic, Glasgow).

Attempts have been made to separate target bacteria from other material prior to culture. For example, EP-A-0489920 describes a process in which antibodies are used to capture bacteria which are separated and subsequently cultured. Separation of target cells from a mixed population using magnetic beads of microspheres is also known, for example from U.S. Pat. No. 4,230,685, EP-A-605003 and P. D. Patel (1994) Microbiological applications of Immunomagnetic techniques in “Rapid Analysis Techniques in Food Microbiology”, Ed P. D. Patel, Blackie Academic & Professional, Glasgow, pp 104-13 1.

Magnetic beads may be coated with antibodies which are specific for particular cell.

When beads are added to a sample, any target cell present will be bound to the surface of the beads. The beads can then be removed from the remainder of the sample using magnetic separation. After separation, the beads including the cells are washed and then taken forward for further investigation. In some instances, this involves culturing the beads to allow any captured microorganisms to reach measurable levels.

New methods for detecting cells by detecting cellular components in a highly sensitive manner mean that the culture times can be reduced significantly, which is a real benefit. One such method is described by Blasco et al. J. Applied Microbiology 1998, 84, 661-666 in which specific assays for bacteria are carried out by using phage mediated release of the enzyme, adenylate kinase. This is then detected in a highly efficient manner using a bioluminescent assay. In this way, low numbers of cells can be detected in a matter of hours.

However, methods of this type are sensitive to contamination. The applicants have found that when attempting to utilise such methods in conjunction with a pre-concentration step, such as those that involve magnetic beads, washing of the beads is rarely effective in removing all possible contaminants that could interfere with the assay for the cellular component. It is believed that support surfaces, such as those found in magnetic beads and in assay plates can attract contaminants such as proteins, which are not then removed easily by washing alone.

The applicants have been working on a method for separating a microorganism from samples containing or suspected of containing said microorganism, said method comprising:

i) contacting said sample with a binding agent for said microorganism, wherein the binding agent is immobilised on a support, and allowing the binding agent to bind said microorganism to form an immobilised complex;
ii) separating the sample from the immobilized complex;
iii) contacting the support with a liquid medium and releasing any microorganisms from the support into the medium; and
(iv) separating the support, if necessary, from the liquid medium.

This method facilitates the detection of microorganisms, where in a further step (v), the presence of microorganisms in particular in the liquid medium is detected.

According to the present invention there is provided a method for detecting a microorganism in a sample containing or suspected of containing said microorganism, said method comprising:

i) contacting said sample with a binding agent for said microorganism, wherein the binding agent is immobilised on a support, and allowing the binding agent to bind microorganism to form an immobilised complex;
ii) separating the sample from the immobilised complex;
iii) contacting the support with a liquid medium and a reagent which removes which eliminates inactivates or inhibits a contaminant that may interfere with a microorganism detection assay; and
iv) detecting microorganisms retained on the support using said microorganism detection assay.

The applicants have found that by using a reagent that effectively removes troublesome contaminants early in the procedure, in the presence of the support, the reliability and efficacy of the detection can be significantly enhanced.

The detection step (iv) may be carried out on the support itself, or, some or all of the microorganisms may be released into a liquid medium, for instance the liquid medium used in step (iii) prior to detection, in which case microorganisms in the liquid medium may alternatively or additionally be detected during step (iv).

In one embodiment, at least some microorganisms are released from the support into the medium during step (iii). In a particular embodiment, the reagent used in step (iii) achieves this release function as well as removing contaminants.

If the microorganisms are released from the support during step (iii), this means that the support may then be separated from the liquid medium prior to the detection of microorganisms in step (iv). The applicants have found that by separating the microorganism from the support, contaminants which may interfere with any subsequent detection assay and which have a tendency to adhere to the surface of the support can also be eliminated more effectively. This means that a wider range of detection assays may be employed, with greater sensitivity.

However, separation is not always essential provided the removal of contaminants in step (iii), which is effectively a purification step has taken place.

Detection assays or methods may take various forms including culturing the cells on a plate. However, detection is more suitably conducted using a cellular assay such as those described in more detail below. The use of the reagent which removes contaminants likely to interfere with the specific assay being used early in the process means that the reliability of the assay is enhanced.

In particular, the liquid medium, in the presence or absence of the support, is passed through a filter which retains either just the support, or where appropriate, the support and any released microorganisms, on its surface. Where both the support and released microorganisms are to be retained, it may be preferable, to avoid clogging problems, if these are carried out on two filters, a preliminary and secondary filter, one which retains the support and one which retains the microorganism.

In particular embodiments, a cellular assay is then conducted on the filter surface containing the support and/or retained microorganisms, or where the filter has simply removed the support, and microorganisms have been released into the liquid medium, on material retained in the filtrate.

During step (iii), the liquid medium and the reagent which eliminates, inactivates or inhibits a contaminant that may interfere with a subsequent detection assay may be added in any order, or they may be added together to the sample. For example the liquid medium may be subject to a purification step, for example one which removes any free protein from the liquid medium is carried out. Removal of proteins means that any reactive proteins such as enzymes which may be present in the sample and which could impact on a detection assay which relies on enzymatic activity are eliminated well before the detection assay begins, so reducing the risk of false positive results.

Thus particular purification steps include a proteolytic step, which may be carried out for example by adding a proteolytic enzyme to the liquid medium.

Alternatively, the liquid medium may be treated with a reagent which inhibits the activity of molecules such as enzymes which may interfere with a later detection assay. Chemical enzyme inhibitors are known in the art, and include substances such as nitrobenzoic acid derivatives such as dithio-bis-nitrobenzoic acid (DTNB), and it is possible that these could be added instead of or in addition to a proteolytic step.

Depending upon the nature of the binding agent, provided this comprises a protein element such as an immunoglobulin, the addition of a proteolytic enzyme may also have the effect of releasing the microorganism from the support into the medium.

In this way, the release and purification steps may be conducted simultaneously. However, additional or alternative steps may be conducted on the liquid medium at this stage to remove other contaminants.

Alternatively or additionally, the immobilised complex may be subject to a culture step after step (iii). During this stage, the microorganism will multiply and cells or colonies may “bud” off of the support, as the binding agent sites become saturated. In this way, microorganism is released into the liquid medium without the need for a specific release reaction, although for maximising sensitivity and speed, it may be desirable to utilise such a step in order to ensure that substantially all of the available microorganism is released.

Detection in step (iv) may be carried out by any convenient method. As described above, in a particular embodiment, the liquid medium and optionally also the support, is passed through a filter, which is able to retain the microorganisms and, if necessary, the support thereon. The pore size of the filter will depend upon the nature of the microorganisms sought and the size of support, but in general, for the detection of bacteria such as Salmonella, the filter size will be less than 1 μm, for instance from 0.2-0.8 μm. Commercially available filters such as those of pore sizes 0.22 μm, 0.45 μm or 0.65 μm are conveniently utilised. For retention of the support and microorganisms a combination of filters, for instance, 1.2 μm, 3 μm, and 0.22-0.8 μm may be used, so that the support is retained on the larger filter, used as a preliminary filter, and the microorganisms are retained on the secondary filter which will have a smaller pore size accordingly.

In a particular embodiment, the filter is one which retains the microorganisms on its surface where they may be directly detected. Suitable filters are generally of a plastics or polymeric material such as polycarbonate, polyethersulphone (PES), polyvinylidene fluoride (PVDF) or cellulose derivatives. They may comprise specific filters or be elements within a filter-bottomed microtitre plate.

The use of such filters for the separation of microorganisms from liquid media forms a further aspect of the invention. Microorganisms separated in this way may be detected using conventional methods.

The filter is suitably washed to remove contaminants including the products of any proteolysis step. Microorganisms captured by the filter may then be detected using a conventional method. If desired, they may be removed from the filter surface prior to detection. For example, the cellular products of microbial lysis (achieved by chemical or biological means) may be drawn through the filter into a vessel below using for example vacuum or positive pressure. In particular the specific bacterial detection method using a combination of specific phage and an assay for adenylate kinase as described herein is carried out on cells retained on the filter.

Suitable microorganisms include fungi, or more particularly, bacteria such as Salmonella, Listeria or E. coli such as toxigenic E. coli.

The binding agent used in step (i) is suitably a specific binding agent for a particular target microorganism, such as a Salmonella, Listeria or E. coli bacteria. Suitable specific binding agents include immunoglobulins such as antibodies or binding fragments thereon. These may be immobilised on the support using conventional methods.

These include (a) direct nonspecific adsorption; (b) covalent coupling via a spacer chemical linkage such as a hydrocarbon chain and (c) by first binding an antibody binding protein such as Protein A or Protein G to the support before application of the binding antibody. In a preferred embodiment, a protein comprising an antibody binding domain and a surface binding domain such as a cellulose binding domain, is applied to the surface, and the binding antibody applied subsequently. Even coverage of the surface is also preferred to avoid “patches” where target organisms may not bind.

A particularly preferred protein for use in attaching an antibody to a nitrocellulose membrane comprises a cellulose binding domain-Protein A conjugate obtainable from Sigma Chemical Co. under the trade name Cellulose binding domain Protein A fusion protein (CBD-Protein A).

Once the binding member is fixed to the surface, remaining binding sites are suitably blocked using a blocking agent such as casein, as is understood in the art.

In some cases, supports such as a magnetic beads which have suitable antibodies already applied are available commercially.

By using a specific binding agent in step (i), some concentration of a particular target microorganism is effectively carried out. This may mean that any microorganism detected in a subsequent step (iv) would be of the target type. However, to avoid the possibility that some non-specific binding has occurred, it may be preferable to utilise a method in step (iv) in which a specific target microorganism, such as a bacteria is detected. One method which allows for specific identification is described in WO9406931, the entire content of which is incorporated herein by reference.

In this method, in essence, a sample is incubated in the presence of a bacteriophage which specifically infects a particular target bacteria, so as to cause lysis of the bacteria. At this point, cellular components are released from the bacteria, and detection of any of these is indicative of the presence of the specific bacteria in the initial sample. The enhanced purification opportunities afforded by the use of the method described above, such as a proteolytic step, is extremely beneficial here, in that it will ensure that no false positives are generated as a result of contaminants which may be retained upon the support.

Assays for a wide variety of cellular components including hormones, enzymes etc. are reported in the art. One cellular component which may be conveniently detected however is the nucleotide adenosine triphosphate or ATP. ATP is conveniently detected using a bioluminescent assay, such as the well known bioluminescent assay based upon the reaction of luciferase and luciferin.

However, in a particular embodiment, the cellular component released on cell lysis which is detected is adenylate kinase. This enzyme catalyses the following equilibrium reaction in cells:

Mg.ATP+AMP [symbol] 2ADP

WO9417202 and WO9602667 describes how the detection of this particular enzyme produces a greatly amplified signal, and the entire content of these documents is incorporated herein by reference.

In brief, adenylate kinase is detected by adding an excess of pure ADP to the sample, so the equilibrium is driven towards the right and ATP is created. This can readily be detected using a variety of assays, but in particular a bioluminescent assay, such as that based upon the reaction of a luciferase enzyme on its substrate luciferin. In the presence of ATP, this interaction occurs and a light signal is generated.

The fact that this type of assay is so sensitive means that low levels of microorganisms can be detected, and therefore the need for extended culture periods can be reduced. This allows the determination of microorganisms and particularly specific target microorganisms to be effected rapidly and accurately. Thus the method described above is highly advantageous in the field of testing of consumer products such as food, and also in clinical or hygiene applications.

However, it is also clear that any contaminating adenylate kinase in the sample may cause false positives to occur. Thus in this particular case, the use of a proteolytic purification step which remove this contaminant and allow the assay to be used more reliably.

The support used in the method described above could be any suitable material as would be understood in the art. Supports will be solid under the conditions of the method, and therefore will generally be of an insoluble material, although supports which may be soluble under certain conditions (such as resins and the like), may be employed. In a particular embodiment, the support is suitably magnetic beads or magnetic nanoparticles, such as those that are readily available commercially, or it may comprise a plate such as an immunoassay plate or a well in such a plate such as a microtitre plate.

Where carried out, the separation of the support from the liquid medium may be achieved using any suitable method. The precise method used will depend upon the precise nature of the support being used. For example, where the support is a microtitre plate or the like, the liquid medium may be removed by pipetting out from the well. Where the support comprises magnetic beads, the liquid medium may be separated by pipetting but also, the magnetic beads may be separated using a filter, with a pore size which is sufficiently large to allow the microorganisms to pass through but which traps the beads. This will vary depending upon the relative sizes of the beads and the microorganisms, but in general, a filter with a pore size of at least 1.2 μm will be sufficient.

In a particularly preferred embodiment, the support comprises beads and in particular magnetic beads. This has implications in terms of the volume of sample required. In order to use beads effectively, a larger sample volume (for example 5-10 ml or more) may be required as compared to say a microtitre plate where small samples (for example 250 μl) are used. However, the applicants have found that this is preferable in accordance with the method of the invention, in order to provide good and reliable results.

If necessary in order to achieve a suitable sample volume or concentration of microorganisms, the sample may be subject to a preliminary incubation or pre-enrichment step, as is conventional in the art. During this step, the sample is mixed with an enrichment broth and then incubated for example in a Stomacher, for a suitable period of time, which may be for example from 1-24 hours. However, the use of a pre-enrichment step means that the potential for requiring further incubation or enrichment after step (iii) above is reduced, and the process is suitably carried out without such a step.

The samples are suitably derived from food samples which are being tested for contamination by microorganisms. In particular, raw meat and processed foods may contain high levels of free adenylate kinase, which will interfere with any microorganism detection assay which detects this component.

In a particular embodiment, the residue on a support which has been separated from the liquid medium is plated out to provide a confirmatory test for the presence of microorganisms. Where the support is a bead for example, this may be effected by streaking the bead onto a culture plate.

Sample preparation methods, carried out prior to step (i) may be necessary depending upon the nature of the sample as well as the nature of the suspected contamination etc. These are generally known in the art, and may include steps such as homogenisation, stomaching, incubation (with or without shaking) or other culture steps.

Kits adapted for conducting the methods described above form a further aspect of the invention. Thus for example, the invention provides a kit for detecting a microorganism in a sample, said kit comprising a support having binding agents for microorganisms immobilised thereon, combined with one or more of

(i) a reagent which eliminates, inactive or inhibit a contaminant, such as a proteolytic enzyme or a chemical enzyme inhibitor; and
(ii) a filter capable of capturing microorganisms. Suitably kits comprise both (i) and (ii).

Other elements useful in the method described above may also be included in the kit. For instance, the kit may further comprises a bacteriophage which specifically infects and lyses a target bacteria, which is used in step (iv) for the specific determination of the target bacteria as described above.

Furthermore, it may further comprises ADP, suitably in pure form, in order to act as a basis for the AK assay described above. Bioluminescent reagents activated by ATP, such as luciferin and a luciferase may also be included in order to allow the specific sensitive AK assay described above to be incorporated into the kit.

The method described above is widely applicable to the detection of a range of microorganisms from a wide variety of samples. However, where for example they are used to detect a specific bacteria such as Salmonella in a food sample, a typical procedure would include the following steps:
(i) starting with a test food sample, allow Salmonella to grow in a liquid culture broth for 10-18 h, preferably with shaking;
(ii) take a portion of the broth and separate Salmonella from the background food particles using a brief centrifugation step;
(iii) capture and concentrate Salmonella from the broth using specific immunomagnetic particles;
(iv) reduce background interference (e.g. adenylate kinase, AK) and assist in release salmonella cells adsorbed to the beads using a proteolytic step;
(v) Separate beads and capture the released salmonella onto a filter followed by a washing step to remove the proteolytic enzyme and its hydrolytic products. Plate the remaining beads onto a solid selective agar medium;
(vi) Carry out specific bacteriophage infection and lytic process to release salmonella-specific AK; and
(vii) Measure the AK using a bioluminescence assay and record the results in terms of a ratio of AK released from the salmonella cells and the background AK.

The invention will now be described by way of example with reference to the accompanying diagrammatic drawings in which:

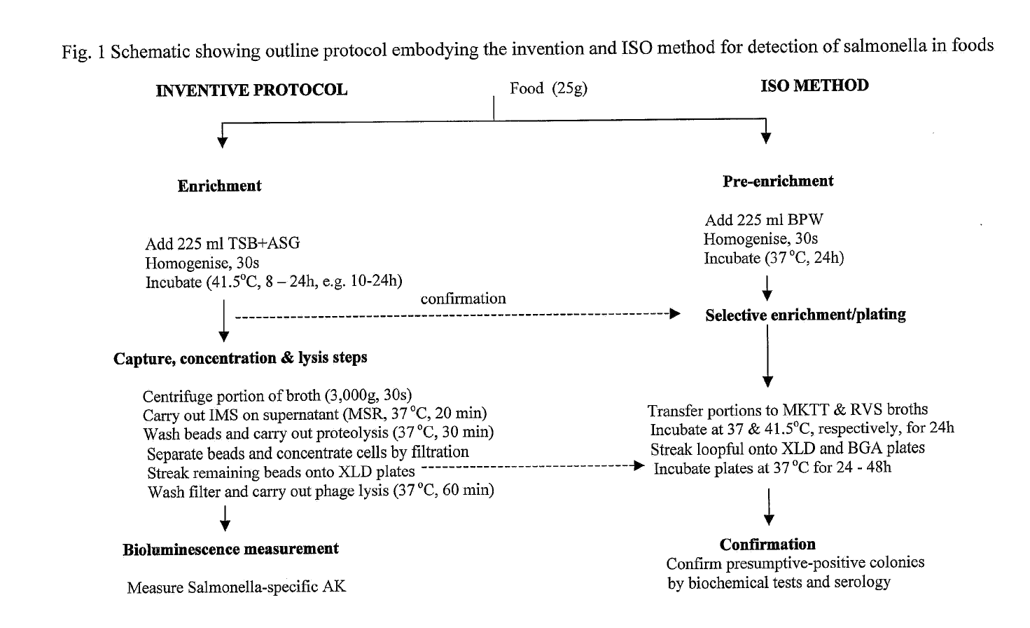

FIG. 1 is a schematic showing outline protocol embodying the invention and ISO method for detection of salmonella in foods; and

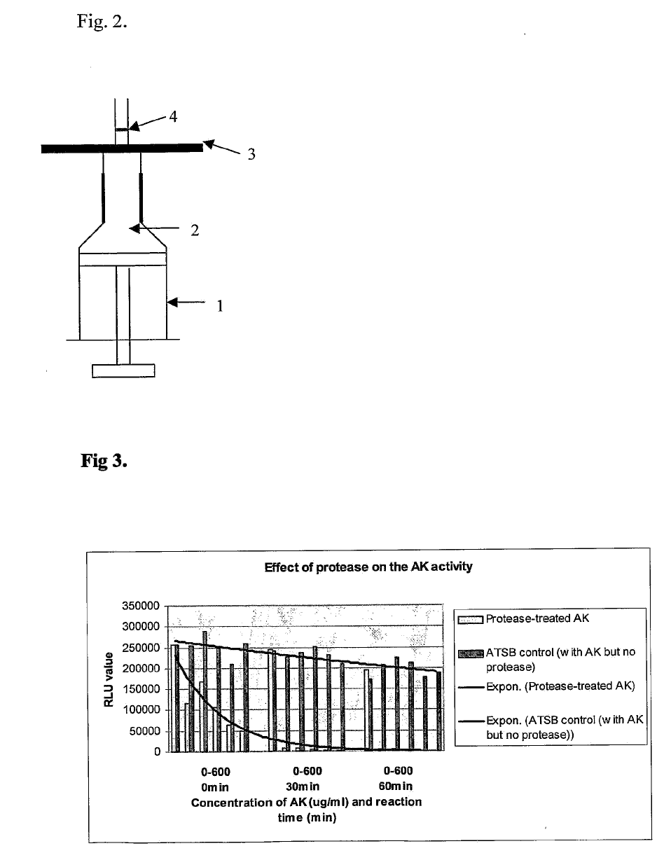

FIG. 2 is a schematic diagram showing an arrangement which may be utilised in order to ensure the release of AK from Salmonella captured on a filter, for assay purposes in accordance with an embodiment of the invention.

FIG. 3 is a graph showing the results of the treatment of with a proteolytic enzyme on the activity of the enzyme adenylate kinase.

### ILLUSTRATIVE EXAMPLE A

### Effect of Protease on Adenylate Kinase (AK) Activity

A range of different concentrations of standard AK was treated with varying concentrations of a broad spectrum protease enzyme for various times (0, 30 and 60 min). The AK assay (see Example 1 step 6) was carried out using ADP (5 min) and luciferase.

The results are shown in FIG. 3. Overall, the protease treatment reduced the AK activity significantly as the reaction time increases from 0 to 60 min, particularly at higher protease concentrations.

### EXAMPLE 1

### Protocol for Detection Assay Compared to ISO Method

### Step 1

A test food sample (25 g) is weighed into a sterile plastic filter bag, to which is added 225 ml of broth medium (TSB+AGS) and the mixture homogenised in a Stomacher for 30 seconds. The sample is then incubated in a shaker-incubator at 41.5° C. and 120 rpm for 10 to 12 hours.

In contrast, for the ISO method, 225 ml Buffered Peptone Water (BPW) is added to a 25 g test food sample, which is then stomached for 30 s and incubated for 18-24 h at 37° C.

### Step 2

After incubation is complete, 12 ml of the sample is transferred to a 15 ml centrifuge tube.

The remaining sample is returned to the incubator without shaking to the 41.5° C. incubator without shaking to be used in a confirmation test as outlined in FIG. 1. Both this, and the sample being treated using the ISO method are then incubated further for a total period of 18-24 h, using the procedures outlined in FIG. 1.

### Step 3

The extracted sample from step 2 is then centrifuged at 3,000 rpm for 30 s. with ‘O’ holding time. 10 ml supernatant is transferred into a fresh centrifuge tube containing 20 μl Salmonella Dynabeads (available from Dynal Norway) and mixed in the Alaska Magnetic Sample Rotator (MSR) at 37° C. for 20 min.

### Step 4

The beads were then washed once (IMS wash) with 1×10 ml warm ATSB in the MSR, and re-suspended in 1 ml of protease solution (100 μg/ml; from Strep. griseus, Sigma 81748) made up in 50 mM PBS (phosphate buffered saline) containing 0.1% glucose and 5 mM MgSO₄. The sample is then vortex mixed and the tubes incubated at 37° C. for 30 minutes.

### Step 5

The samples are then again vortex mixed and the beads separated to sides of the tube. 2×495 μl samples of liquid are then removed from the tube and transfer each to separate 0.45 μm filters (for T1 and T2 readings; defined below) to concentrate the cells on the filter. [The remaining 10 μl sample in the tube is plated out onto xylose lysine deoxycholate (XLD) agar as a confirmatory step.]

### Step 6

Each filter is washed with 25 ml of warm 50 mM PBS containing 0.1% glucose and 5 mM MgSO₄ and the effluent discarded, with the exception of approximately 2 ml which is retained for analysis of background AK activity using the method generally described in Blasco et al. supra. In summary in this instance, sample (100 μl) and ADP (50 μl) are incubated for 5 minutes after which a luciferin/luciferase mixture (50 μl) is added and luminescence measure immediately.

Washing is continued, if necessary, until the effluent readings are similar to the wash buffer. This value is referred to as T₀. At this stage, all effluent may be discarded.

### Step 7

As a result, each sample is associated with 2 filters. To one of the filter (T1 background value), 200 μl of a Salmonella phage diluent (ATSB) is added. The other filter (3) (FIG. 2) is arranged above an inverted syringe (1) (FIG. 2) containing 200 μl of phage solution (2) such that the solution soaks the filter, with a phage solution meniscus (4) above the level of the filter (3). The complete assembly (filter and syringe) is left to stand upside down for 60 min at 37° C.

### Step 8

Each phage solution is then collected by flushing through the filter into a sterile Eppendorf tube. The contents of the tube are vortex mixed and AK activity of all the samples (T1 and T2 values) measured using the general method of Blasco et al supra. and with the following quantities
Sample . . . 100 μl
AD . . . 50 μl
Incubate for 5 min then add:
Luciferin/luciferase . . . 50 μl
Immediately measure luminescence

If the result of T2 is higher than T1, then the food sample is contaminated with Salmonella.

Samples tested using the method of the invention gave similar results to those tested during the ISO method, but in a shorter timescale. The results were confirmed by the confirmatory test conducted as described in step 5.

### EXAMPLE 2

### Modified Protocol for Detection Assay of Salmonella Using the Method of the Invention

A test food sample (25 g) is weighed into a sterile plastic filter bag, to which is added 225 ml of broth medium (BPW+Tween 80) and the mixture homogenised in a Stomacher for 30 seconds. The sample is then incubated in a incubator at 37° C. and 120 rpm for at least 16 hours to produce a pre-enriched sample.

After incubation is complete, 12 ml of the sample is transferred to a 15 ml centrifuge tube. This is then centrifuged at 2,500-3,000 rpm for 30 seconds with ‘O’ holding time. 10 ml supernatant is transferred into a fresh centrifuge tube containing 20 μl Salmonella Dynabeads (available from Dynal Norway), the tube is capped, and then placed in a rack of an Alaska Magnetic Sample Rotator (MSR) without the magnet in place, and rotated at 5 rpm at 37° C. for 20 min. A magnet is then introduced into the rack of the MSR and rotation continued at 5 rpm for a further 5 minutes at 37° C.

The rack is then removed from the MSR, the cap removed from the tube and the supernatant pipetted off to waste, with the magnet in place.

A wash medium (such as Alaska Wash Medium A available from Alaska Diagnostics Limited (UK) (10 ml), pre-warmed to 37° C. is added to each tube, and these are then inverted to ensure the beads are in suspension before being returned to the rack. The magnet is then introduced and the rack rotated again in the MSR at 37° C. and 5 rpm for 5 minutes. The supernatant is then pipetted off once more, and 0.5 ml of prewarmed (37° C.) protease solution as described in Example 1 step 4 (in Phosphate buffered saline,PBS,pH 7.4) is added.

The tubes are then vortex mixed at high speeds for 5 seconds and immediately aliquots (2×240 μl from each tube) are transferred to wells in a microtitre filter plate, having a pore size of less than 3 μm, for instance commercially available filters of pore sizes 0.22 μm, 0.45 μm or 0.65 μm, 1.2 μm and 3 μm.

[The residual beads can optionally then be transferred or streak plated onto XLD plates, which are incubated for 24 hours at 37° C. as a confirmatory test.]

The filter plate is then connected to a vacuum manifold to draw liquid through, although the filters are not allowed to completely dry. Each filter is washed by addition of 200 μl of PBS solution, which is substantially completely removed using the vacuum, a wash step which is repeated from 7 to 10 times.

As a result, each sample is associated with 2 filter wells. To one of the filter well (T1 background value), 100 μl of a Salmonella phage diluent (ATSB), pre-warmed to 37° C., is added, and to the other (T2), 100 μl of a Salmonella phage solution, also pre-warmed to 37° C. The plate is sealed under film and incubated above a white microtitre plate for 60 minutes at 37° C.

The contents of the microtitre filter plate are then drawn through into the corresponding wells of the white microtitre plate below using vacuum. Control wells for AK, broth and phage are then set up by addition of the relevant moiety to clean wells in the white filter plate.

The contents of the plate are then assayed for adenylate kinase with a luciferase/luciferin bioluminescent signalling system, as is known in the art. The luminescence from each well is measured using a luminometer, and the ratio of the values obtainable from the T1 and T2 wells can be used to determine whether the sample is contaminated with Salmonella.

Using this method, results can be obtained within 18 hours, (as compared to 72 hours for the conventional culture method) and with accuracy typically around 95%.

1-35. (canceled)

36. A method for separating a microorganism from food samples containing or suspected of containing said microorganism, said method comprising:
(a) mixing the food sample with an enrichment broth and incubating the broth so as to multiply microorganisms contained therein;
(b) contacting said sample with a binding agent for said microorganism, wherein the binding agent is immobilized on a support, and allowing the binding agent to bind said microorganism to form an immobilized complex;
(c) separating the sample from the immobilized complex;
(d) contacting the support with a liquid medium to form a liquid sample; and
(e) passing said liquid sample through a filter which retains microorganism on the surface of the filter.

37. A method according to claim 36, wherein microorganism retained on the filter is a bacteria and is detected by incubating the bacteria with a bacteriophage, which infects the target bacteria and causes lysis thereof, and thereafter, detecting a cellular component released as a result of the lysis.

38. A method according to claim 37, wherein the released cellular component is adenylate kinase and is detected by adding an excess of ADP and detecting ATP produced.

39. A method according to claim 38, wherein the ATP is detected using a bioluminescent assay.

40. A method for detecting a microorganism in a sample containing or suspected of containing said microorganism, said method comprising:
(i) contacting said sample with a binding agent for said microorganism, wherein the binding agent is immobilized on a support, and allowing the binding agent to bind microorganism to form an immobilized complex;
(ii) separating the sample from the immobilized complex;
(iii) contacting the support with a liquid medium and a reagent which eliminates, inactivates or inhibits a contaminant that may interfere with a microorganism detection assay; and
(iv) detecting microorganisms retained on the support using said microorganism detection assay.

41. A method according to claim 40, wherein at least some microorganisms retained on the support are released into the liquid medium during step (iii), the support is separated from the liquid medium prior to step (iv), and microorganisms retained in the liquid medium are detected in step (iv).

42. A method according to claim 40, wherein the reagent used in step (iii) eliminates, inactivates or inhibits free protein.

43. A method according to claim 42, wherein the reagent used causes proteolysis.

44. A method according to claim 43, wherein the proteolysis is carried out by adding a proteolytic enzyme to the liquid medium.

45. A method according to claim 42, wherein the reagent used is a chemical enzyme inhibitor.

46. A method according to claim 40, wherein, after step (ii), the immobilized complex is cultured.

47. A method according to claim 46, wherein the culture is continued for long enough to allow at least some bacteria to separate from the support.

48. A method according to claim 40, wherein in step (iv), released microorganisms are captured on a filter and detected thereon.

49. A method according to claim 48, wherein the microorganisms are lysed to release cellular contents and a cellular component is detected in the assay.

50. A method according to claim 40, wherein the binding agent used in step (i) is specific for a particular target microorganism.

51. A method according to claim 50, wherein the binding agent is an antibody or binding fragment thereof, which specifically binds a target microorganism.

52. A method according to claim 40, wherein a specific target microorganism is detected in step (iv).

53. A method according to claim 40, wherein the target microorganism is a bacteria.

54. A method according to claim 53, wherein the bacteria is Salmonella, Listeria or toxigenic E. coli.

55. A method according to claim 53, wherein the bacteria is detected by incubating the bacteria with a bacteriophage, which infects the target bacteria and causes lysis thereof, and thereafter, detecting a cellular component released as a result of the lysis.

56. A method according to claim 55, wherein the released cellular component is adenylate kinase.

57. A method according to claim 56, wherein adenylate kinase is detected by adding an excess of ADP and detecting ATP produced.

58. A method according to claim 57, wherein the ATP is detected using a bioluminescent assay.

59. A method according to claim 40, wherein the support comprises magnetic beads.

60. A method according to claim 40, wherein the support comprises a plate or a well in a plate.

61. A method according to claim 40, wherein residues on the support separated in step (iv) are plated out to provide a confirmatory test.

62. A kit for detecting a microorganism in a sample, said kit comprising a support having binding agents for microorganisms immobilized thereon, combined with a reagent which eliminates, inactivates or inhibits a contaminant of a microorganism detection assay and/or a filter capable of capturing microorganisms.

63. A kit according to claim 62, wherein the reagent is a proteolytic enzyme or a chemical enzyme inhibitor.

64. A kit according to claim 62, which further comprises a bacteriophage that infects and lyses a target bacteria.

65. A kit according to claim 62, which further comprises ADP.

66. A kit according to claim 65, which further comprises a bioluminescent system that is activated by ATP.

67. A kit according to claim 66, wherein the said bioluminescence system comprises luciferin and luciferase.

68. A method for separating a microorganism from a liquid sample containing it, said method comprising passing said liquid sample through a filter which retains said microorganism on a surface of the filter.

69. A method according to claim 68, wherein the microorganism retained on the filter is a bacteria and is detected using a specific bacteria detection method.

70. A method according to claim 69, wherein the bacteria is detected by incubating the bacteria with a bacteriophage that specifically infects the bacteria and causes lysis thereof, and thereafter, detecting a cellular component released as a result of the lysis.

71. A method according to claim 70, wherein the released cellular component is adenylate kinase.

72. A method according to claim 71, wherein adenylate kinase is detected by adding an excess of ADP and detecting ATP produced.

73. A method according to claim 72, wherein the ATP is detected using a bioluminescent assay.
